(19) United States
(12) Patent Application Publication
(10) Pub. No.: US 2004/0047892 A1
(43) Pub. Date: Mar. 11, 2004

(54) Filler composition for soft tissue augmentation and reconstructive surgery

(76) Inventors: Eric Andre Desrosiers, Outremont (CA); Abdellatif Chenite, Kirkland (CA); Cyril Chaput, Montreal (CA); Matthew Shive, Montreal (CA)

Correspondence Address: David S Resnick, Nixon Peabody, 101 Federal Street, Boston, MA, 02110, US

(21) Appl. No.: 10/416,942

(22) PCT Filed: Nov. 15, 2001
(86) PCT No.: PCT/CA01/01622

(51) Int. Cl. A61K 38/39

(52) U.S. Cl. 424/423; 514/2; 514/55

(57) ABSTRACT

The present invention relates to a filler composition for soft-tissue augmentation and reconstructive surgery comprising an effective amount of an injectable thermo-gelling solution comprising 0.1 to 5.0% by weight of chitosan or collagen or a derivative thereof; and 1.0 to 20% by weight of a salt of polyol or sugar selected from the group consisting of mono-phosphate dibasic salt, mono-sulfate salt and a mono-carboxylic acid salt of polyol or sugar. The solution is stable and turns into a gel within a tem-perature range from 20 to 70° C. The gel has a cosmetically acceptable consistency for providing a mechanical support to surrounding soft tissues once injected therein. The composition can thus be used as filler for soft tissue augmentation and reconstructive surgery.

### BACKGROUND OF THE INVENTION

1. (a) Field of the Invention

The invention relates to a cosmetic composition for use as filler for soft tissue augmentation and reconstructive surgery.

2. (b) Description of Prior Art

A wide variety of injectable materials have been used to fill out lines and creases caused by ageing, gravity and sun exposure. Those materials for soft tissue augmentation can be categorized in relation to their origin: synthetic, xenogeneic, homogeneic and autogeneic, and subdivided according to their longevity in the patient as, temporary, undefined or permanent.

The oldest filler on the market, and still the most widely used filler is xenogeneic collagen (e.g. Zyderm & Zyplast, of bovine origin, from Collagen Corp. or the porcine Fibrel, from Mentor Corp.). The effect of this filler is temporary. It disappears within a few months, depending on the patient and site of injection. In addition, about 3% of the population is allergic to collagen, requiring that skin tests be performed before the treatment. Furthermore, their mammalian origin leaves the risk of transmissible disease. The use of autologous collagen, or collagen obtained from cell cultures is emerging as an alternative to the xenogeneic product.

The technique of micro-lipoinjection, injecting autologous fat sub-cutaneously, has a long clinical history and is recognized as a safe procedure (Coleman, W. P., Dermatol Clin, 17:723-7, 1999). Microlipoinjection has been used since 1986 by cosmetic surgeons to fill cosmetic defects with autologous fat tissues obtained from liposuction at another site on the patient. Likewise, surgical transplantation of autologous fat has been used for an even longer period, with the same goal. However, the effect is even less durable than that of collagen. Furthermore, autologous (autogenic) transplants require two surgical interventions: one to remove tissue from the patient, and another to implant it at another site.

Other fillers are available in some markets. Retsylane is an injectable form of hyaluronic acid. It does not require a pre-treatment skin test, and lasts on average about 2 months more than collagen. Artecoll™, a suspension of 40 μm polymethylmetacrylate (PMMA) beads and collagen offers a reportedly much longer useful life. It is also painful to inject, and requires a skin test. Liquid Silicon has been used in the past, and caused inflammation and granulomas. Isolagen (Isolagen Technology), which uses the patients' own expanded dermal fibroblasts to thicken the dermis, requires complex manipulations.

Aside from providing only short-term correction, none of those fillers forms a continuous solid at the injection site, and can thus hardly provide the mechanical support required for large defects or reconstructive surgery. In those cases, it is usually required to surgically implant molded forms to act as mechanical support.

Microdispersions (solid particles in a liquid) have been proposed as injectables for soft-tissue repair and augmentation (Scopelianos et al. U.S. Pat. No. 5,599,852). They proposed a bioabsorbable microdispersion consisting of a liquid polymer comprising lactone units with a particulate component made of synthetic homopolymers. Soft-tissue repair and augmentation in animals was again described by Scopelianos et aL (U.S. Pat. No. 5,824,333) who inject an injectable bioabsorbable liquid copolymer, generally copolymer of lactones such caprolactone, trimethyl carbonate, etc.

Dispersion of beads or particles in a lubricative suspension, solution, fluid or gel was proposed by Lawin (U.S. Pat. No. 5,792,478).

Soft-tissue augmentation of mammals can be augmented by injecting keratin into the soft-tissue such as the bladder or urethral tissue (Smith, U.S. Pat. No. 5,712,252).

An injectable composition of elastin and collagen and a biocompatible carrier was also presented by Janzen et al. (U.S. Pat. No. 5,705,488) for soft tissue augmentation.

Biocompatible ceramic microspheres in a lubricious gel carrier were also proposed by Hubbard (U.S. Pat. No. 5,922,025). Other biodegradable and injectable microspheres were proposed as injectable for soft-tissues (WO99/11196).

Dunn et al. (U.S. Pat. No. 5,278,202) proposed thermosetting solid-forming materials for injection in soft-tissues. Such materials consisted in a polymer with a solvent or a curing agent, and formed solid implants in situ. The effectiveness of such injectable implants for soft-tissue substance augmentation appears quite disputable due to the solid formation in situ.

Naughton (U.S. Pat. No. 6,234,284) proposed a cell-free human secreted extracellular matrix for injection into a skin defect.

The use of a biocompatible polymer dissolved in a biocompatible solvent was reported by Greff et al. (U.S. Pat. No. 6,231,613). Cellulose acetate, ethylene vinyl alcohol and polyacrylates were the preferred polymers.

It would be highly desirable to be provided with an injectable filler that could provide a durable correction, especially if it provides a substantial mechanical support to the surrounding soft tissues.

It would be highly desirable to be provided with an injectable filler that could form in situ a gel-like implant to provide a durable correction.

It would be highly desirable to be provided with an injectable filler that could form in situ a gel-like implant to provide an augmentation of a soft-tissue substance, thus enabling a durable volume or thickness increase.

### SUMMARY OF THE INVENTION

One aim of the present invention is to provide an injectable filler composition that could provide a durable correction or augmentation, especially if it provides a substantial mechanical support or volume or thickness increase to the surrounding soft tissues.

Another aim of the present invention is to provide a method for filling out lines and creases caused by ageing, gravity and sun exposure.

In accordance with the present invention there is provided a polysaccharide-based gel which comprises: a) 0.1 to 5.0% by weight of chitosan, collagen or a derivative thereof; and b) 1.0 to 20% by weight of a salt of polyol or sugar selected from the group consisting of mono-phosphate dibasic salt, mono-sulfate salt and a mono-carboxylic acid salt of polyol or sugar; wherein said solution is stable and turns into a gel within a temperature range from 20 to 70° C., said gel having a cosmetically acceptable consistency for providing a mechanical support to surrounding soft tissues once injected therein.

The salt may be any of the following or in any of the following combination: a) a mono-phosphate dibasic salt selected from the group consisting of glycerol, comprising glycerol-2-phosphate, sn-glycerol 3-phosphate and L-glycerol-3-phosphate salts; b) a mono-phosphate dibasic salt and said polyol is selected from the group consisting of histidinol, acetol, diethylstilbestrol, indole-glycerol, sorbitol, ribitol, xylitol, arabinitol, erythritol, inositol, mannitol, glucitol and a mixture thereof; c) a mono-phosphate dibasic salt and said sugar is selected from the group consisting of fructose, galactose, ribose, glucose, xylose, rhamnulose, sorbose, erythrulose, deoxy-ribose, ketose, mannose, arabinose, fuculose, fructopyranose, ketoglucose, sedoheptulose, trehalose, tagatose, sucrose, allose, threose, xylulose, hexose, methylthio-ribose, methylthio-deoxy-ribulose, and a mixture thereof; d) a mono-phosphate dibasic salt and said polyol is selected from the group consisting of palmitoyl-glycerol, linoleoyl-glycerol, oleoyl-glycerol, arachidonoyl-glycerol, and a mixture thereof; and e) glycerophosphate salt is a selected from the group consisting of glycerophosphate disodium, glycerophosphate dipotassium, glycerophosphate calcium, glycerophosphate barium and glycerophosphate strontium.

A preferred gel in accordance with one embodiment of the present invention is selected from the group consisting of chitosan-β-glycerophosphate, chitosan-α-glycerophosphate, chitosan-glucose-1-glycerophosphate, and chitosan-fructose-6-glycerophosphate.

Solid particulates or water-soluble additives may be incorporated within said polysaccharide-based gel prior to the gelation.

Drugs, polypeptides or non-living pharmaceutical agents may be incorporated within said polysaccharide-based gel prior to the gelation.

Living microorganisms, plant cells, animal cells or human cells may be encapsulated within said polysaccharide-based gel prior to the gelation.

The gel may be formed in situ subcutaneously, intra-peritoneally, intramuscularly or within the substances of biological connective tissues, organ walls or parts, body conduits or cavities, eye cul-de-sac, etc.

In accordance with the present invention there is also provided a method for producing the composition described above, which comprises the steps of: a) dissolving a chitosan, collagen or a derivative thereof within an aqueous acidic solution of a pH from about 2.0 to about 5.0 to obtain an aqueous solution having a concentration of 0.1 to 5.0% by weight of a chitosan, collagen of a derivative thereof; b) dissolving 1.0 to 20% by weight of a salt of polyol or sugar into the aqueous solution of step a) to obtain an injectable thermogelling solution, wherein said salt is selected from the group consisting of mono-phosphate dibasic salt, mono-sulfate salt and a mono-carboxylic acid salt, wherein said an injectable thermogelling solution has a concentration of 0.1 to 5.0% by weight of a chitosan, collagen or a derivative thereof, and a concentration of 1.0 to 20% by weight of a salt of a polyol or sugar, and has a pH from about 6.4 to about 7.4.

This method may further comprises a step c) after step b), of heating said polysaccharide-based gel solution at a solidifying temperature ranging from about 20° C. to about 80° C. until formation of a polysaccharide gel.

A pharmaceutical agent may be added to the polysaccharide gel solution of step b).

The method may further comprises a step i) after step b), of dispensing for gelation the polysaccharide-based gel solution into a desired receiver, either in a mold or within a tissue, an organ or a body cavity.

The aqueous acidic solution may be prepared from at least one organic or inorganic acid selected from the group consisting of acetic acid, ascorbic acid, salicylic acid, phosphoric acid, hydrochloric acid, propionic acid, and formic acid.

The gelling point of the polysaccharide-based gel solution may be adjusted such that the polysaccharide-based gel solution may be kept in a stable ungelled liquid form at a temperature ranging from about 0° C. to about 20° C.

The solidifying temperature is preferably ranging from about 20° C. to about 60° C., more preferably about 37° C.

The molecular weight of chitosan is preferably ranging from about 10,000 to 2,000,000.

Solid particulate additives may be added to the polysaccharide-based gel solution of step b).

The polysaccharide-based gel solution may be introduced within an animal or human body by injection or endoscopic administration, and gelled in situ at a temperature of about 37° C.

In accordance with the present invention there is also provided the use of the polysaccharide-based gel for producing biocompatible degradable materials used in cosmetics, pharmacology, medicine and/or surgery.

The gel may be incorporated as a whole, or as a component, into implantable devices or implants for repair, reconstruction and/or replacement of tissues and/or organs, either in animals or humans.

The gel may be used as a whole, or as a component of, implantable, transdermal or dermatological drug delivery systems.

The gel may be used as a whole, or as a component of implants or drug delivery systems.

Still in accordance with the present invention, there is provided the use of the cosmetic or surgical composition defined above as filler for soft tissue augmentation and reconstructive surgery, or for producing biocompatible degradable materials.

The complementary polymer is a non-ionic water-soluble polysaccharide, a methylcellulose, a hydroxyalkyl cellulose, a poly(alkylene oxide) or a poly(alkylene glycol), or a derivative or a copolymer thereof.

For the purpose of the present invention the following terms and expressions are defined below.

The term “Polysaccharide-based gel solution” is intended to mean a polysaccharide solution in a stable ungelled liquid form at a temperature ranging from about 0° C. to about 15° C. which can be gelled or changed to a gel state when heated at the gelling temperature.

The term “gelling temperature” is intended to mean any temperature ranging from about 20° C. to about 80° C., preferably between 37° C. to about 60° C., and more preferably at about the physiological temperature or 37° C.

The expression “salts of polyols or sugars” is intended to mean mono-phosphate di-basic salts, mono-sulfate salts and mono-carboxylic acid salts of polyols or sugars.

The present invention include method of forming different gelled materials, those materials being either molded (customized shapes, tubes, membranes, films . . . ) or formed in situ within biological environments (filling of tissue substances).

In a preferred embodiment, the chitosan/organo-phosphate aqueous solution has a pH above the pKa of chitosan and turn into solid gel upon thermal stimulation. This polysaccharide gel can be used as a carrier for drugs or as a non-living therapeutics delivery systems, as substituting materials for tissues and organs and as encapsulants for living cells or microorganisms. Chitosan/organo-phosphate gel matrices are rapidly formed at temperatures between 30 to 60° C. Chitosan/organo-phosphate aqueous systems are used as injectable filling materials, injected and gelled in situ for filling and repairing tissue substances.

Glycerol-2-phosphate, glycerol-3-phosphate and glucose-1-phosphate based salts are the preferred disclosed salts in accordance with the present invention.

Chitosan/polyol- or sugar-phosphate and chitosan/polyol- or sugar-sulfate gels can be applied also to surgical reconstructive and regeneration uses and drug delivery purposes. They provide thermally reversible or irreversible bioerodible polymeric gels with biologically well-known and compatible components for a broad range of medical/biotechnological applications.

In another preferred embodiment, the composition comprises at least one fatty acid, that is selected preferably in a group consisting of palmitate, stearate, myristate, palmitoleate, oleate, vaccenate, linoleate, and the like, and their acyclic, cyclic, heterocyclic, aromatic ester derivatives containing at least one moiety selected from the group consisting of hydroxy, acyloxy, aryloxy, amino, sulfhydryl, sulfonate, sulfate, phosphonate, phosphate, bis-, tris- and poly- phosphonates and phosphates, phosphatidyl, nucleosides, oligosaccharides, polysaccharides, polyols, and the like.

In one preferred embodiment, the fatty acid is mixed with an appropriate metabolically absorbable liquid vehicle to reduce viscosity and allow injectability at room temperature.

The fatty acid solution may comprise a metabolically absorbable liquid vehicle selected in a group consisting of water, alcoholic solvent, alkylene glycol, poly-alcohol, and the like. The metabolically absorbable liquid vehicle is preferably selected in a group consisting of ethanol, isopropyl alcohol, ethylene glycol, glycerol, and the like.

In one preferred embodiment, the solution comprises oleoate and palmitate. The solution may be under gel or solid form at low to room temperatures, e.g. 20 degrees Celsius and below, but may become more or less viscous liquids at higher temperatures, e.g. above 35-40 degrees Celsius.

### BRIEF DESCRIPTION OF THE DRAWINGS

FIG. 1 illustrates the tissue-bulking effect of a chitosan hydrogel filler formulation of the present invention, 6 months after sub-cutaneous injection in a human;

FIG. 2 illustrates the tissue-bulking effect of a fatty acid filler formulation of the present invention, 28 days after sub-cutaneous injection in a rat; and

FIGS. 3A to 3D illustrate histopathology images from BST-InPod™ injections in rats (Saffranin-O/Fast Green staining) from day 2 at 4X and 40X, (FIGS. 3A and 3B, respectively), and from day 5 at 4X and 40X (FIGS. 3C and 3D, respectively).

### DETAILED DESCRIPTION OF THE INVENTION

In accordance with the present invention, there is provided thermoforming chitosan-based and fatty acid-based solutions, and uses thereof. Both compositions are easily injectable, gels in situ and provide substantial mechanical support to the surrounding soft tissues. The solution remains liquid during injection and gels after injection as it reaches body temperature.

The biodegradability of the thermo-gelling chitosan-based solution can be adjusted to be resistant to biodegradation so as to be effective for a long-term, or even permanent, correction, as desired. This combination of characteristics makes possible the long-lasting smoothing of small wrinkles or the correction of pronounced defects. The possibilities of this material fulfill the more demanding needs of reconstructive surgery, bridging the gap to a field that usually require surgically implanted polymeric forms.

The thermo-gelling chitosan-based solution forms a gel that has mechanical properties compatible with the needs of both cosmetic and reconstructive surgery, if required, sufficiently soft for use in cosmetic surgery for correction of fine skin defects or sufficiently rigid for correction of large defects or use in reconstructive surgery.

Chitosan is dissolved in acidic aqueous solutions so as to obtain clear aqueous chitosan solutions having pH levels within the range 4.3 to 5.6. The chitosan solutions can be sterilized through filtering or steam-autoclaving, and stored at low positive temperature (4° C.). The organo-phosphate component is added to the chitosan solution, preferably at low positive temperature (4° C.), then the aqueous chitosan/organo-phosphate mixture is gelated thermally, through an endothermal mechanism, within the temperature range from 30 to 60° C. Once formed the resulting chitosan/organo-phosphate gels are thermally stable upon heating even up to 180° C. (in autoclave), particularly in cell culture medium. Bioencapsulation within chitosan/organo-phosphate gels is obtained by incorporating the living cells within the ungelated aqueous chitosan/organo-phosphate solution at a low temperature (4° C.). Then the temperature of the resulting mixture chitosan/organo-phosphate/cells is raised to and maintained at 37° C. where the gelation occurs in ˜1 hour. organo-sulfates or mono-carboxylic acid salt of polyols or sugars play a similar role than organo-phosphates.

Chitosan and its derivatives are relatively inexpensive and commercially available materials and represent an attractive group of biocompatible and degradable polymers. They have solid or solution properties that can be modified by changing their chemical composition and/or physico-chemical characteristics. The deacetylation degree and molecular weight have been shown to greatly influence the solution properties, enzymatic degradability and biological activity. Chemical modifications, for instance, have been proposed to neutralize or modify chitosan chains by incorporating carboxylic acid, acetate, glutamic acid, carboxymethyl or sulfate groups. Chemical cross-linking (anhydride, glutaraldehyde, glutamate succinimide-PEG . . . ) of chitosan macromolecules induces covalent bonds to create branched or grafted networks.

Physical gelation of chitosan and its derivatives can be obtained through different techniques:

a) neutralization (NaOH, KOH, NH₄OH . . . ) which induces hydrogen bonding between chitosan chains;

b) ionic complexation with divalent anions (borate, molybdate, polyphosphate, sulfate salts and sulphated macromolecules . . . ) which induces pure electrostatic interactions; and

c) complexation with anionic surfactants (sodium alkyl sulfate . . . ), which induces electrostatic interactions and surfactant-surfactant hydrophobic interactions.

In accordance with the present invention there is proposed a new gelation mechanism that combines hydrogen bonding, electrostatic interactions and chitosan-chitosan hydrophobic interactions. It can only be achieved through complex interactions between chitosan macromolecules, water molecules and mono-phosphate dibasic salts of polyols or sugars.

Polyols are frequently added to compositions for improving gel properties. Sorbitol and mannitol are currently used as tonicity enhancing agents. Glycerol and polyethylene glycol are proposed as plasticizers. Polyols (-ol: glycerol, sorbitol . . . ) and sugars (-ose: fructose, glucose, galactose . . . ) were used as thermal stabilizing agents for proteins in solutions. Depending on the selected molecules, they were found to make or break structuring of water, create hydrogen bonding, electrostatic or hydrophobic interacting, and present endothermic transitions (. Polyols and sugars stabilize proteins to heat denaturation through their structuring effect on water and the strengthen of hydrophobic interactions.

Beta-glycerophosphate disodium or calcium salt, or glycerol-2-phosphate disodium or calcium salt, is a well studied molecule in biological sciences. It is considered as a substrate for alkaline phosphatase (AL). glycerophosphate is widely used as a cell culture medium supplement for culturing cells isolated from musculo-skeletal tissues, and has been shown to induce or maintain the synthesis of specific matrix components when delivered to bone/cartilage cells in culture. Gelation of chitosan will occur with any grade or purity glycerophosphate while encapsulation of living biologicals would require cell culture tested glycerophosphate. Alpha-glycerophosphate disodium or calcium salt, or glycerol-3-phosphate disodium or calcium salt, is also an organic salt of biological importance. Glycerophosphate salts are precipitated from glycerophosphoric acids that are obtained through the hydrolysis of lecithin, a well-know biological molecule and phosphatides of eggs, soybean and fishes. Glycerophosphoric acids are present under two isomeric structures, the alpha and beta, wherein the beta-glycerophosphoric acid is optically inactive and the alpha-glycerophosphoric acid is optically active. Glycerophosphoric acid is physiologically active compound, being involves in the catabolism of carbohydrates. Glycerophosphoric acid is currently available under disodium, calcium, magnesium, dipotassium, strontium and barium salts, having a relatively strong basic character. Both alpha- and beta-glycerophosphate salts are inexpensive readily available sources of organic mono-phosphate dibasic salts among the polyol or sugar phosphate salts.

Solubilization of chitosan in aqueous solutions requires the protonation of the amine groups of the chitosan chains, which is reached within acidic aqueous solutions having a pH ranging from 3.0 to 5.0. When solubilized, chitosan remains soluble until a pH about 6.2. Neutralization of acidic chitosan solutions by alkali results in a pH increase as well as a de-protonation of the amine groups. Neutralization of acidic chitosan solutions to a pH above the pKa of chitosan at about 6.3-6.4 results in OH—HN and O—HN interchains and water-chitosan hydrogen bonds, which- induce a hydrated three-dimensional network, a chitosan gel. At pH above 6.3-6.4, chitosan solutions result systematically into chitosan gels at a normal temperature range (0-60° C.). However, admixing of an organo-phosphate to a chitosan aqueous solutions increases the pH of the chitosan/organo-phosphate solutions which remain ungelled and liquid for long periods of time even at pH above 6.5, and up to 7.2. This neo-neutral chitosan/organo-phosphate aqueous solutions (pH 6.5-7.2) will gel when stimulated by an adequate temperature. The time of gelation is controlled by the temperature. For example, a chitosan/organo-phosphate solution that gelates in about 30 minutes at 37° C., needs only about 2 minutes at 60° C. to form a gel.

The mechanism of gelation as well as the gel characteristics has been expected to be similar for all chitosan/organo-phosphate systems. Thus, the gelation of chitosan/β-glycerophosphate solutions, which has been investigated in more details, can be considered as typical example.

Another important characteristic is related to the injectability and in vivo gelation of chitosan/β-glycerophosphate solutions.

In chitosan/organo-phosphate gels, organo-phosphate anions contribute to the cross-linking of chitosan macromolecule chains, but not in the same way as the pure ionic cross-linking that takes place during the gelation of chitosan by inorganic divalent anions, such as sulfate, oxalate, phosphate or polyphosphate (pyrophosphates, metaphosphates or tripolyphosphates). A chitosan aqueous solution turns into gel instantaneously in presence of inorganic divalent anions and independently of the solution pH value. Furthermore, the elevation of temperature constitutes an unfavorable factor for the gelation of this kind of systems. In contrast, the gelation of chitosan/organo-phosphate solution depends on both, the final pH of chitosan/organo-phosphate solution and the temperature. Every solution of chitosan/organo-phosphate can not be gelled, at any temperature, as long as its pH remains below 6.45, and every solution of chitosan/organo-phosphate with pH above 6.45 can be prepared at 20° C., without immediate gellation and can be stored for long time at 4° C. without turning to gel. At 37° C. only the chitosan/organo-phosphate solutions with pH above 6.9 can be gelled more or less rapidly. It is expected that the presence of organo-phosphate molecules in chitosan solutions directly affects electrostatic interactions, hydrophobic interactions and hydrogen bonds of chitosan chains. Thus, the main interactions involved in the formation of chitosan/organo-phosphate gels become essentially: 1) chitosan/chitosan interchain hydrogen bonding; 2) chitosan/organo-phosphate electrostatic attractions between the ammonium groups of macromolecule chains and the phosphate group of organo-phosphate molecules; 3) chitosan-chitosan hydrophobic interactions induced through the structuring action of the polyol or sugar parts on water molecules. The structuring action of the polyol parts on water reduces the chitosan-water interactions and therefore enhances the chitosan-chitosan interactions. The nontrivial aspect of such a gelation originates essentially from the later polyol-water induced chitosan hydrophobic attractions, which are enhanced upon increasing temperature (temperature-controlled gelation). At low temperatures, chitosan-water strong interactions protect the hydrated chitosan macromolecules against aggregations. Removal upon heating of the sheath of water molecules favors and strengthens chitosan-chitosan interactions, and hence induces the macromolecules association. However, the gelation would never occur if the two first attractions are fully unoperational within the chitosan/organo-phosphate solution. This explains the pH-dependence that still governs the temperature-controlled gelation of chitosan/organo-phosphate systems. Although such electrostatic attractions are present, the phosphate groups can not be the unique cross-linker agent of chitosan chains due to non-compatible stearic hindrance. This significantly differentiates this gelation mechanism from the pure ionic gelation of chitosan by phosphates or polyphosphates divalent anions. A pure ionic cross-linking would not be temperature-controlled or stimulated.

This type of temperature-controlled pH-dependant gelation is specifically induced by organic mono-phosphate dibasic salt in chitosan solution, however it may be induced as well by other organic salts such as mono-sulfate salts of polyols or sugars, such as polyol-sulfate or sugar-sulfate, or mono-carboxylic acid salts of polyols or sugars. For example, in accordance with the present invention, a chitosan/glucose-1-sulfate solution is expected to gel so as a chitosan/glucose-1-phosphate solution does.

It is also an aim of the present invention to provide an aqueous chitosan/organo-phosphate solution that can be formed and stored at low temperature (4° C.) and transformed at physiological temperatures into three-dimensional stable chitosan/organo-phosphate gel. It includes nontoxic biocompatible components for mammalian or human environments with both components and processes having low toxicity effects towards living biologicals and preserving the cellular viability. The gel also provides good mechanical/handling performances for long periods of time at the physiological temperature and in physiological aqueous media containing amino-acid, ions and proteins. Chitosan derivatives may be selected as well as to process chitosan/organo-phosphate gels, and comprise N, O-substituents of chitosan.

The expression “organo-phosphates (salt)” refers herein, without limitation, to mono-phosphate dibasic salts of polyols or sugars, such as polyol-phosphate dibasic salts or sugar-phosphate dibasic salts. Organo-sulfates (salt) also refer herein to mono-sulfate salts of polyols or sugars, such as polyol-sulfate salts or sugar-sulfate salts. The preferred organo-phosphate salts may be selected from mono-phosphate dibasic salts of glycerol, including glycerol-2-phosphate, sn-glycerol 3-phosphate and 1-glycerol-3-phosphate salts (alpha-glycerophosphate or beta-glycerophosphate), mono-phosphate dibasic salts of histidinol, acetol, diethylstilbestrol, indoleglycerol, sorbitol, ribitol, xylitol, arabinitol, erythritol, inositol, mannitol, glucitol, palmitoyl-glycerol, linoleoyl-glycerol, oleoyl-glycerol or arachidonoyl-glycerol, and mono-phosphate dibasic salts of fructose, galactose, ribose, glucose, xylose, rhamnulose, sorbose, erythrulose, deoxy-ribose, ketose, mannose, arabinose, fuculose, fructopyranose, ketoglucose, sedoheptulose, trehalose, tagatose, sucrose, allose, threose, xylulose, hexose, methylthio-ribose or methylthio-deoxy-ribulose. Other mono-salts of interest (sulfate, carboxylate) may be derived from the same polyols or sugars.

The expression “glycerophosphate or glycerophosphate” refers herein to both alpha-glycerophosphate and beta-glycerophosphate isomers. Alpha-glycerophosphate is indistinctively referred for glycerol-3-phosphate (all optical eniantomers) while beta-glycerophosphate is similarly referred for glycerol-2-phosphate.

The expression “three-dimensional” refers herein to the fact that the polymeric solution is simultaneously gelated and shaped by the mold wherein the solution was initially poured. Gels can be produced in glass or plastic bechers, dishes, tubes or between two plates so as to obtain any expected shapes.

The expression “in situ gelation” refers herein to the formation of chitosan/organo-phosphate gels by injecting the liquid chitosan/glycerophosphate solution within specific sites of mammalian or human environments, e.g. any tissues (muscles, bone, ligaments, cartilages) and organs. Gelation in situ allows complete and precise filling of tissue defects or body cavities. The gelation of the chitosan/organo-phosphate mixture is induced by the physiological temperature. Gelling and gelation are used herein without any distinction.

The expression “endothermal gelation” refers herein to the thermal mechanism of the chitosan/organo-phosphate solution that enables the solution to gelate upon standing at the desired temperature. Induction of sol to gel transitions of chitosan/organo-phosphate systems requires energy via, for example, the temperature.

The expression “cells or cellular matters” refers herein to living biologicals, such as isolated cells, cellular dispersion, cell aggregates, cell spheroids or cells adhered to solid microspheres particles, that are encapsulated within the chitosan/organo-phosphate gels.

The expression “in situ forming” refers herein to the procedure of administrating the ungelated chitosan/organo-phosphate liquid solution to a body site (e.g. connective tissues, body conduits, articular cavities, fractures, bone defects . . . ), and inducing and ensuring within the body site at the physiological temperature a complete gelation of the polysaccharide solution into a gel.

Formation of Chitosan/organo-phosphate Gels

The selected organo-phosphate salt was herein glycerophosphate, but similar results were reached with other mono-phosphate dibasic salts of polyols or Sugars. Chitosan in powder form is dissolved in an aqueous acidic solution until the occurrence of a clear solution is obtained. The proportion of chitosan varies from 0.5 to 5.0% w/v, preferentially from 1.0 to 3.0% w/v. The pH of the aqueous chitosan solution ranges from 4.5 to 5.5. Aqueous chitosan solutions can be sterilized either by filtration with in-line sterile filters (0.22 micrometer) or by steam-autoclaving (120° C.). Sterilization of the chitosan/glycerophosphate gels can not be filtered due to the viscosity or steam-autoclaved due to the thermal sensitivity, but can be performed by gamma-irradiation or reached through strictly sterile procedures. Freshly-prepared aqueous chitosan solutions are stored preferably at low positive temperature (4° C.). Glycerophosphate felt in fine powder form is added to, and dissolved within, the aqueous chitosan solution at a temperatures ranging from 4 to 15° C., preferentially 10° C. When a clear homogeneous chitosan/glycerophosphate aqueous solution with a pH ranging from 6.5 to 7.2 is attained, the said solution is poured into the desired receiver, and hold to appropriate temperature to gel. Glycerophosphate felt in form of aqueous solution may be also used.

Depending on their final pH, the chitosan/glycerophosphate solutions are expected to lead either to thermally reversible or irreversible gel. Reversible gels arise from chitosan/glycerophosphate solutions having a pH comprising between 6.5 and 6.9, while the irreversible gels originate from chitosan/glycerophosphate solutions having a pH above 6.9. The nature of the acid that is used for the acidic chitosan solutions does not influence fundamentally the sol to gel transition of the chitosan/glycerophosphate system. The final pH within a chitosan/glycerophosphate solution is dependent upon the pH of the water/acid solution as well as the chitosan and glycerophosphate concentrations. As chitosan and glycerophosphate are two alkaline components, they tend to increase the pH of the acidic solution wherein they are dissolved. Concentrations in chitosan and glycerophosphate can be balanced to reach the appropriate pH of the chitosan/glycerophosphate solution, while taking into consideration the solubility limit of both components, and particularly the one of chitosan.

Three-Dimensional Monolithic Gels

The selected organo-phosphate salt was herein glycerophosphate, but similar results were reached with other mono-phosphate dibasic salts, monosulfate salts or monocarboxylate salts of polyols or sugars. The receiver or mold filled with chitosan/glycerophosphate solution is heated at a temperature ranging from 30 to 60° C., preferentially 37° C. The gelation of chitosan/glycerophosphate solution at 37° C. can be performed within a common cell culture incubator. The solution is maintained at the desired temperature until it turns into a gel after a period that ranges from some days to a week (at 30° C.) to few minutes (at 60° C.). At 37° C., the gelation of chitosan/glycerophosphate solution occurs in 1 hour approximately. Once a three-dimensional chitosan/glycerophosphate gel is formed, the said gel is demolded and washed in distilled water. Chitosan/glycerophosphate gels remain stable and keep their three-dimensional shape even at high temperature, 120° C. (in autoclave).

Chitosan/glycerophosphate based compositions may also comprise additional water-soluble ingredients. For example, the said Chitosan/glycerophosphate solution may comprise an additional polymer selected in a group consisting of cellulose, methyl cellulose and derivatives, hydroxyalkyl cellulose and derivatives, water-soluble vinyl polymers, poly(alkylene glycol) and copolymers, poly(alkylene oxide) and copolymers, mono-functional poly(ethylene glycol), and any mixture thereof. This novel polymer component may significantly change some composition properties, but does not alter its “gel forming capacity”.

The compositions may comprise additionally a water-soluble chemical agent having a pKa between 6.0 and 8.2 such as a water soluble inorganic salts, and preferably dibasic salts, and for example water-soluble dibasic phosphate, sulfate or carbonate salts. It may also be a biological buffer such as a organic salt or an amino-acid, or a sequence of amino-acids.

In Situ Formation of Gels

The selected organo-phosphate salt was herein glycerophosphate, but similar results were reached with other mono-phosphate dibasic salts, monosulfate salts or monocarboxylate salts of polyols or sugars. In situ gelation of the chitosan/glycerophosphate solution can be conducted by dispensing the solution from a hypodermic syringe. If needed, the solution may be pre-gelated (initiate the thermal gelation) by keeping the syringe and chitosan/glycerophosphate solution at desired temperature, ideally 37° C., until the first signs of gelation appear. The ready-to-gel chitosan/glycerophosphate mixture is then administrated so as to fill tissue defects or cavities and complete in situ the gelation process (at 37° C.). Injection of chitosan/glycerophosphate solutions is however limited by the viscosity of the solutions, which controls the injectability, or extrudability of the solutions. Needles having a gauge of 27 and below are ideal materials for injection of such gel solution. Body cavities and tissue defects act as recipients for the solution, but the liquid materials remain in an open aqueous environment. The conformability and diffusability of the chitosan/glycerophosphate solutions is dependent upon the solution and material properties. Increased viscosity results in formation in situ of more compact and less conformable gels.

Other Fillers

In the present invention, the filler composition may be composed of a fatty acid mixtures.

In another preferred embodiment, the said composition comprises one or more natural or unnatural saturated and mono- or poly-unsaturated fatty acids, that are selected preferably in a group comprising palmitate, stearate, myristate, palmitoleate, oleate, vaccenate, linoleate, and the like, and their acyclic, cyclic, heterocyclic, aromatic ester derivatives containing one or more groups such as hydroxy, acyloxy, aryloxy, amino, sulfhydryl, sulfonate, sulfate, phosphonate, phosphate, bis-tris- and poly- phosphonates and phosphates, phosphatidyl, nucleosides, oligo-saccharides, polysaccharides, polyols, and the like, and a mixture thereof.

In one preferred embodiment, the said fatty acid component is mixed with an appropriate metabolically absorbable liquid vehicle to reduce viscosity and allow injectability at room temperature.

The fatty acid solution may comprise a metabolically absorbable liquid vehicle selected in a group comprising water, alcoholic solvents, alkylene glycols, poly-alcohols, and the like. The metabolically absorbable liquid vehicle is preferably selected in a group comprising ethanol, isopropyl alcohol, ethylene glycol, glycerol, and the like, and any mixture thereof.

In one preferred embodiment, the said solution comprises oleoate and palmitate. The said solution may be under gel or solid form at low to room temperatures, e.g. 20 degrees Celsius and below, but may become more or less viscous liquids at higher temperatures, e.g. above 35-40 degrees Celsius.

Injections to Soft-Tissues

It is intended that such filler compositions for soft-tissue augmentation and reconstructive surgery are fully injectable, and can be applied to various methods of use.

In the present invention, a method of treating urinary incontinence comprising the step of injecting the said filler composition into the area of the urethral sphincter, said composition having a bulking action into said sphincter.

A method of breast augmentation comprises the step of injecting the said filler composition into the breast, said composition being use to increase the tissue volume.

A method of cosmetic treatment of wrinkles comprises the step of injecting the said filler composition into the soft tissue in or around the face, said composition having a cosmetically acceptable consistency for providing a mechanical support or a volume or thickness increase to surrounding soft tissues.

A method of load bearing tissue augmentation comprises the step of injecting the said filler composition between said load bearing tissue and a load-exerting medium.

A method of treating acne scars or viral pock marks comprises the step of injecting the said filler composition into the soft tissue underlying said scar or pock mark.

A method of changing the contours of a nose comprises the step of injecting the said filler composition into the soft tissue of the nose.

More generally, in the present invention it is proposed a method of augmenting the volume or thickness of soft-tissues comprising the step of injecting the said filler composition into the soft tissue substance. A method of operating plastic corrections can comprise the step of injecting the said filler composition into soft tissue substances or tissue cavities to create plastic corrections. A method of operating reconstructive or restorative surgeries can comprise the step of injecting the said filler composition into soft tissue substances, body cavities or conduits, organ walls or parts to create a reconstructive or restorative action.

The filler compositions is generally injected through an orifice of gauge number above 13, more preferably a gauge number above 22. They are injected through a needle, catheter or trocar. Such compositions can be injected during the course of an endoscopic procedure, or percutaneously.

The filler compositions can be composition is prepared by pre-heating at a temperature between 20 and 45° C. before injection.

The filler compositions can be stored and supplied in a sealed vial or bottle, or in a closed hypodermic syringe.

These compositions can be a part of, or incorporated in, a soft-tissue augmentation kit devoted to healthcare professionals.

Depending upon its composition, the filler compositions can be stored under specific conditions, such as at a temperature below 5° C. before being used, and even solid frozen before being used.

The present invention will be more readily understood by referring to the following examples, which are given to illustrate the invention rather than to limit its scope.

### EXAMPLE I

### Typical gelation of a chitosan/organo-phosphate system

Experiment 1:

Typical experiment was carried out by dissolving 0.2 g of chitosan in 10 ml of aqueous acetic acid solution (0.1M). The pH of the acetic acid solution has been beforehand adjusted to 4.0 by adding droplets of potassium hydroxide solution (1M). The 2% (w/v) chitosan solution so obtained had a pH of about 5.6. Then, 0.800 g of glycerophosphate disodium salt pentahydrate were added to and dissolved in the chitosan solution at 10° C. The pH of the resulting homogeneous liquid mixture becomes 7. This mixture was disposed in a glass scintillation vial in the incubator at 37° C. for 2 hours, enough time to achieve bulk-gelation process. The resulting bulk gel was immersed in renewed baths of distilled water in order to remove the excess of glycerophosphate salt.

A similar result was reached when the glycerophosphate disodium salt (or glycerol-2-phosphate disodium salt) was replaced by the alpha-glycerophosphate disodium salt (or glycerol-3-phosphate disodium salt).

Experiment 2:

A homogenized chitosan/glycerophosphate solution was prepared as in Experiment 1 and disposed in a dual gel caster having a glass plates gel sandwich with a 1.6 mm interspaces, and the system was kept in an oven at 37° C. The formation of a gel membrane was reached within 2 hours and the membrane was unmolded from the gel caster.

Experiment 3:

A 0.110 g of fumed silica under solid particle form (AEROSIL) was dispersed within a solution prepared by dissolving 0.200 g of chitosan in 10 ml of aqueous acetic acid solution. A 0.800 g of glycerophosphate disodium salt pentahydrate was added to the chitosan-silica dispersion. The resulting composition was disposed in a glass scintillation vial in water bath kept at 37° C. The gelation of the chitosan/glycerophosphate component was observed within 2 hours, and the chitosan/glycerophosphate gel includes dispersed solid silica particles.

Experiment 4:

A 0.200 g of chitosan was dissolved in acetic acid solution as in Experiment 1. A 1.239 g of glucose-1-phosphate disodium salt tetrahydrate was added and dissolved so as to reach a clear chitosan/glucose-1-phosphate solution. This chitosan/glucose-1-phosphate solution placed in a glass scintillation vial was maintained at 37° C. The Sol to Gel transition occurs at 37° C. within 3 hours. The resulting bulk gel was immersed in renewed baths of distilled water in order to remove the excess of glucose-phosphate salt.

The experiment was conducted as described in Experiment 4 except that the 1.239 g of glucose-1-phosphate salt was replaced by 0.100 g of fructose-6-phosphate disodium salt dihydrate.

### EXAMPLE II

### Effect of Composition On pH of Solution and Occurrence of Gelation

A mother acidic solution made of a Water/Acetic acid was prepared for all experiments. The pH of this mother acidic solution was adjusted to 4.0. High molecular weight (M. w. 2,000,000) Chitosan powder was added and dissolved in a volume of the mother acidic solution so as to produce Chitosan solutions having Chitosan proportions ranging from 0.5 to 2.0% w/v (Table 1). Table 1 reports the measured pH for the different samples.

TABLE 1
Chitosan Aqueous Solutions and pH levels

| | | | | |
|---|---|---|---|---|
| Chitosan conc. (w/v) | 0.5 | 1.0 | 11.5 | 2.0 |
| PH of Chitosan Sol. | 4.68 | 4.73 | 5.14 | 5.61 |

Glycerophosphate was added to the chitosan solutions and induces a pH increase. Table 2 shows the effect of glycerophosphate concentration on different chitosan solution. The concentration of glycerophosphate ranges from 0.065 to 0.300 mol/L. The chitosan/glycerophosphate solutions in glass vials were maintained at 60 and 37° C., and bulk and uniform gelation was noted within 30 minutes at 60° C. and 6 hours at 37° C. (Table 2). Chitosan and beta-glycerophosphate components individually influence the pH increase within the aqueous solutions, and consequently influence the Sol to Gel transition. As well as the dissolved materials, the initial pH of the mother water/acetic acid solution would also influence the Sol to Gel transition, but this potential effect seems to be limited by the counter-action of the chitosan solubility, which depends on the pH of the solution.

TABLE 2
Gelation of Chitosan/Glycero phosphate Corn positions

| Chitosan conc. | 1.5 | | | 2.0 | | |
|---|---|---|---|---|---|---|
| (w/v) | | | | | | |
| PH of Chitosan Sol. | 5.14 | | | 5.61 | | |
| GP conc. (mol/L) | 0.130 | 0.196 | 0.260 | 0.130 | 0.196 | 0.260 |
| PH of Chitosan-GP | 6.64 | 6.83 | 6.89 | 6.78 | 6.97 | 7.05 |
| Sol. | | | | | | |
| Gelation | | | | | | |
| 60° C. | <30 min. | <30 min. | <30 min. | <30 min. | <30 min. | <30 min. |
| 37° C. | No | No | No | No | <6 hrs | <6 hrs |

### EXAMPLE III

### Biological Response to the Chitosan Hydrogel

The biological properties of chitosan have historically been very well characterized through multiple medical applications. However, the novelty of this thermoforming gel system and its in-situ gelling capabilities called for new biocompatibility studies.

A complete toxicity study has been carried on- the chitosan thermogel (95DDA). The following safety tests were performed under good laboratory practices (GLP) either on the gel per se, or on gel extracts. It has been conducted in compliance with the OECD (Organization for Economic Cooperation and Development) guidelines for testing of chemicals, Paris 1997, and ISO10993-1, Biological evaluation of medical devices standard, 1997.

The test article was found to be non-mutagenic according to a Reverse Mutation Assay with E. coli and S. typhimurium (Ames test), at all dilution levels for both strains, with and without metabolic activation. Also, the results from a second genotoxicity test indicated that the gel did not induce a statistically significant increase in the percentage of Chinese Hamster Ovary Cells (CHO) with chromosomal aberrations at all the dilutions tested, both with and without metabolic activation, when compared to the controls. This is also confirmed by the results of a Mouse Micronucleus Test, where the gel was found to be not genotoxic, at dose levels up to 400X the expected maximum human dose.

A saline extract of the gel was found to be hemocompatible, as it did not cause hemolysis of rabbit blood. It was also non-cytotoxic for L929 mouse fibroblasts monolayers in a direct contact test.

The test article was found to cause negligible irritation response in an Intracutanous Reactivity Test in rabbits, a response similar to the one caused by the blank extract. Finally, the gel in cottonseed oil extract and in saline extract was found to be non-sensitizing in a Skin Sensitization test in Guinea Pigs (aka Maximization test).

Implantation biocompatibility studies were performed. The chitosan thermogel has been administered by sub-cutaneous injections into rats and dogs. Single-dose tests and multiple-dose weekly injections tests were used in both species. In all cases, after 14 weeks, the implants could be retrieved during the necropsy at the site of implantation, without obvious signs of degradation. In another, long term study, the chitosan-gel implants could still be retrieved after 14 months of subcutaneous implantation in rats. At least 80% of the material could be retrieved at this time-point, illustrating the long resilience and effective life of the product.

In all implantation cases, there was no evidence of toxicity related to the product. Subcutaneous injection of the gel in beagle dogs at 5 escalating dose levels (9-90 mg/kg), representing at least 325 times the dose expected to be used in humans for cosmetic injections demonstrated no evidence of systemic toxicity at any dose level. In-life observations and post-necropsy gross pathological examinations revealed no treatment-related effects, aside from the bulking effect at the site of injection, which corresponds to the gelling site.

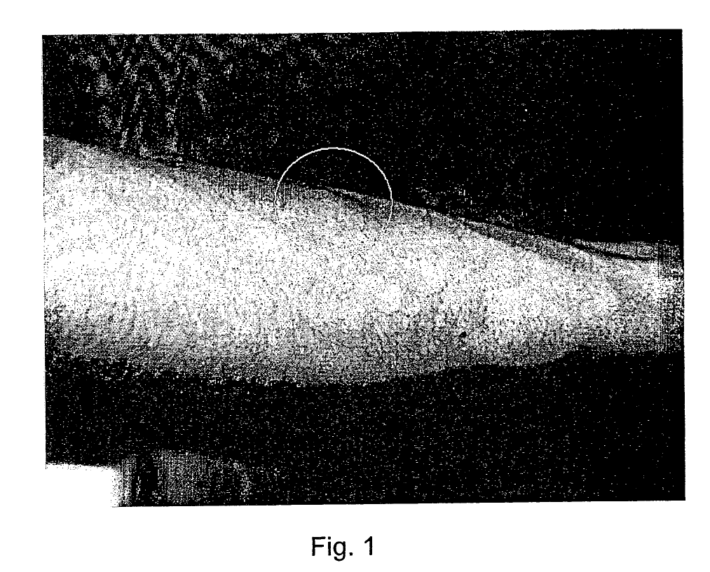

Small 0.1 ml doses of the chitosan thermo-gel (1.5% w/v chitosan 95DDA and 4% w/v B-GP) were also injected sub-cutaneously in the forearm of human volunteers. This human tolerance test showed that the product forms a solid at the site of injection, does not cause noticeable adverse reactions, and provides a lasting bulking effect still visible six months after implantation (ongoing study, 6 month is the latest observation timepoint, See FIG. 1).

### EXAMPLE IV

### Use of a Fatty-Acid Based Filler

A filler formulation can be made of a mixture of fatty acids, such as 84% w/w oleic acid and 14% w/w palmitic acid. The fatty acids are weighed, combined in a container, warmed to melt the components, and mixed. The solution can be sterilized by an appropriate method, preferably by filtering the warm solution through 0.2 μmm filter. This product, stored at or below room temperature, can be used by first warming it up slightly above the melting point of the mixture (35°-39° C.), using warm tap water or another moderate source of warmth. The liquefied solution is then drawn from the vial with a syringe fitted with a fine needle (26G).

A study has been performed on rats to evaluate the tissue-bulking capacity of this filler, its short-term inflammatory response as well as its acute systemic toxicity. For this purpose, Sprague-Dawley Rats have been injected subcutaneously at 30X the dose expected to be used for human facial cosmetic application (2.5 ml). Animals were sacrificed at 2, 5 and 28 days, and samples were processed for histology.

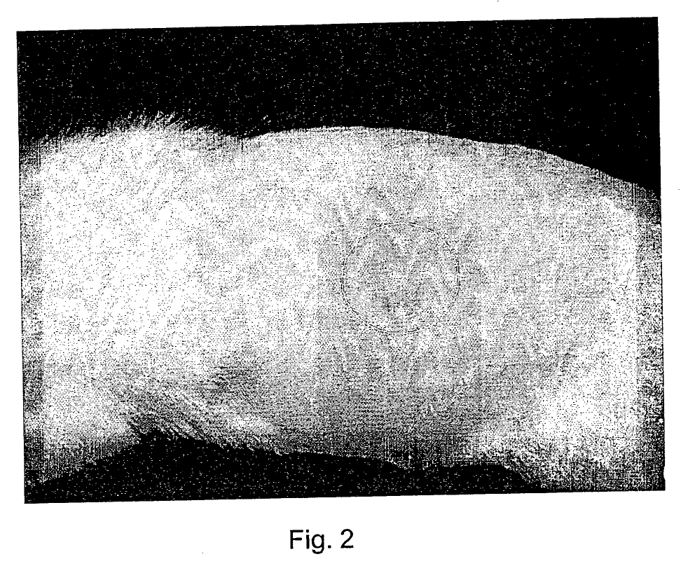

In life observations demonstrated that rats appeared normal and showed no material related gross abnormalities or deaths up until the time of sacrifice. At all time points, shaved injection sites appeared normal and without external indications of inflammation. A clearly defined disk-shaped bump under the skin at all sites of injection was attributed to the test material, and remained present throughout the study (see FIG. 2).

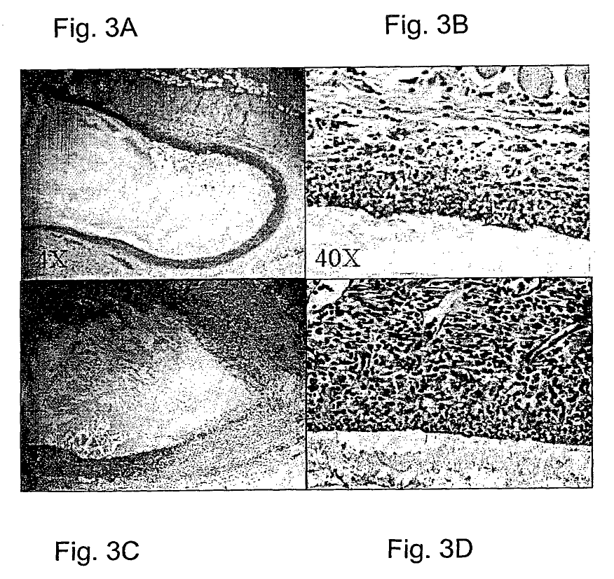

Histologically after 2 days, the injection site was easily identified, and there existed a well defined zone of inflammatory cells including neutrophils, macrophages and some fibroblasts on the outer perimeter of the material. Some necrosis was observed, as was the apparent uptake of fatty acids by phagocytes. The InPod material itself appeared well formed and stable. See FIGS. 3A and 3B.

The 5 day implants resembled the 2 day samples, except that there was an obvious proliferation of fibroblasts and endothelial cells. Some collagen formation was noted, as was neovascularization. The fatty acids implant showed some degeneration on the ends, but appeared stable (See FIGS. 3C and 3D.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A filler composition for soft-tissue augmentation and reconstructive surgery comprising an injectable thermoforming solution comprising:
a) 0.1 to 5.0% by weight of chitosan or collagen or a derivative thereof; and
b) 1.0 to 20% by weight of a salt of polyol or sugar selected from the group consisting of mono-phosphate dibasic salt, mono-sulfate salt and a mono-carboxylic acid salt of polyol or sugar;
wherein said solution is stable and turns into a solid gel within a temperature range from 20 to 70° C., said gel having a acceptable consistency for providing a mechanical support or a volume or thickness increase to surrounding soft tissues once injected therein.

2. The composition of claim 1, wherein said salt is a mono-phosphate dibasic salt of glycerol selected from the group consisting of glycerol-2-phosphate, sn-glycerol 3-phosphate and L-glycerol-3-phosphate salts.

3. The composition of claim 1, wherein said salt is a mono-phosphate dibasic salt and said polyol is selected from the group consisting of histidinol, acetol, diethylstilbestrol, indole-glycerol, sorbitol, ribitol, xylitol, arabinitol, erythritol, inositol, mannitol, and glucitol or a mixture thereof.

4. The composition of claim 1, wherein said salt is a mono-phosphate dibasic salt and said sugar is selected from the group consisting of fructose, galactose, ribose, glucose, xylose, rhamnulose, sorbose, erythrulose, deoxy-ribose, ketose, mannose, arabinose, fuculose, fructopyranose, ketoglucose, sedoheptulose, trehalose, tagatose, sucrose, allose, threose, xylulose, hexose, methylthio-ribose, methylthio-deoxy-ribulose, and a mixture thereof.

5. The composition of claim 1, wherein said salt is a mono-phosphate dibasic salt and said polyol is selected from the group consisting of palmitoyl-glycerol, linoleoyl-glycerol, oleoyl-glycerol, and arachidonoyl-glycerol, or a mixture thereof.

6. The composition of claim 1, wherein said solution is selected from the group consisting of chitosan-β-glycerophosphate, chitosan-α-glycerophosphate, chitosan-glucose-1-glycerophosphate, and chitosan-fructose-6-glycerophosphate.

7. The composition of claim 1, wherein said gel is formed in situ sub-cutaneously, intra-peritoneally, intramuscularly or within the substance of biological connective tissues, organ walls or parts, body conduits or cavities, eye cul-de-sac.

8. The composition of claim 1, wherein said solution comprises an additional polymer selected in a group consisting of cellulose, methyl cellulose or a derivative thereof, hydroxyalkyl cellulose or a derivative thereof, a water-soluble vinyl polymer, a poly(alkylene glycol) and a copolymer thereof, a poly(alkylene oxide) and a copolymer thereof, and a mono-functional poly(ethylene glycol), or a mixture thereof.

9. The composition of claim 1, wherein said solution comprises a water-soluble chemical agent having a pKa between 6.0 and 8.2.

10. The composition of claim 1, wherein said solution comprises a water-soluble phosphate or carbonate salt.

11. A method for producing a composition as defined in claim 1, which comprises the steps of:
a) dissolving a chitosan, collagen or a derivative thereof within an aqueous acidic solution of a pH from about 2.0 to about 5.0 to obtain an aqueous solution having a concentration of 0.1 to 5.0% by weight of a chitosan, collagen or a derivative thereof;
b) dissolving 1.0 to 20% by weight of a salt of polyol or sugar into the aqueous solution of step a) to obtain an injectable thermo-gelling solution, wherein said salt is selected from the group consisting of mono-phosphate dibasic salt, mono-sulfate salt and a mono-carboxylic acid salt, wherein said an injectable thermo-gelling solution has a concentration of 0.1 to 5.0% by weight of a chitosan, collagen or a derivative thereof, and a concentration of 1.0 to 20% by weight of a salt of a polyol or sugar, and has a pH from about 6.4 to about 7.4.

12. The method of claim 11, which further comprises after step b), a step of:
c) heating the injectable thermo-gelling solution of step b) at a solidifying temperature ranging from about 20° C. to about 80° C. until formation of a gel.

13. The method of claim 11, wherein a pharmaceutical agent is added to the injectable thermo-gelling solution of step b).

14. The method of claim 11, wherein said aqueous solution is prepared from at least one organic or inorganic acid selected from the group consisting of acetic acid, ascorbic acid, salicylic acid, phosphoric acid, hydrochloric acid, propionic acid, and formic acid, or a mixture thereof.

15. The method of claim 11, wherein said salt is a mono-phosphate dibasic salt of glycerol is selected from the group consisting of glycerol-2-phosphate, sn-glycerol 3-phosphate and L-glycerol-3-phosphate salts.

16. The method of claim 11, wherein said salt is a glycerophosphate salt selected from the group consisting of glycerophosphate disodium, glycerophosphate dipotassium, glycerophosphate calcium, glycerophosphate barium and glycerophosphate strontium.

17. The method of claim 11, wherein said salt is a mono-phosphate dibasic salt of a polyol, and said polyol is selected from a group consisting of histidinol, acetol, diethylstilbestrol, indoleglycerol, sorbitol, ribitol, xylitol, arabinitol, erythritol, inositol, mannitol, and glucitol, or a mixture thereof.

18. The method of claim 15, 16 or 17, wherein said salt is a mono-phosphate dibasic salt of a sugar, and said sugar is selected from a group consisting of fructose, galactose, ribose, glucose, xylose, rhamnulose, sorbose, erythrulose, deoxy-ribose, ketose, mannose, arabinose, fuculose, fructopyranose, ketoglucose, sedoheptulose, trehalose, tagatose, sucrose, allose, threose, xylulose, hexose, methylthio-ribose, methylthio-deoxy-and ribulose, or a mixture thereof.

19. The method of claim 11, wherein said salt is a mono-phosphate dibasic salt of a polyol, and said polyol is selected from the group consisting of palmitoyl-glycerol, linoleoyl-glycerol, oleoyl-glycerol, and arachidonoyl-glycerol, or a mixture thereof.

20. The method of claim 11, wherein said salt is a mono-phosphate dibasic salt and said phosphate is selected from the group consisting of a phosphate disodium, phosphate dipotassium, phosphate calcium, phosphate barium and phosphate strontium.

21. The method of claim 11, wherein said injectable thermogelling solution is kept in a stable ungelled liquid form at a temperature ranging from about 0° C. to about 20° C.

22. The method of claim 12, wherein the gel is a thermo-irreversible gel when the pH of said injectable thermo-gelling solution is >6.9.

23. Use of a composition as defined in claim 1 as a filler for soft tissue augmentation and reconstructive surgery.

24. Use of a cosmetic composition as defined in claim 1 for producing biocompatible degradable materials.

25. A filler composition for soft-tissue augmentation and reconstructive surgery comprising an injectable thermo-forming solution comprising at least one fatty acid.

26. The filler composition of claim 25, wherein the fatty acid is selected from the group consisting of palmitate, stearate, myristate, palmitoleate, oleate, vaccenate and linoleate, or the like, and their acyclic, cyclic, heterocyclic, aromatic ester derivatives containing at least one moiety selected from the group consisting of hydroxy, acyloxy, aryloxy, amino, sulfhydryl, sulfonate, sulfate, phosphonate, phosphate, bis-, tris- and poly-phosphonates and phosphates, phosphatidyl, nucleosides, oligosaccharides, polysaccharides, and polyols, or the like.

27. The composition of claim 25, wherein said fatty acid is mixed with an appropriate metabolically absorbable liquid vehicle, to reduce viscosity and allow injectability at room temperature.

28. The composition of claim 25, wherein said fatty acid is mixed with a liquid vehicle selected from the group consisting of water, alcoholic solvents, alkylene glycols, and poly-alcohols, or the like.

29. The composition of claim 25, wherein said fatty acid is mixed with liquid vehicle selected from the group consisting of ethanol, isopropyl alcohol, ethylene glycol, and glycerol, or the like, or a mixture thereof.

30. The composition of claim 25, wherein said solution comprises at least palmitate and oleoate.

31. The composition of any one of claims 1 to 10 and 25 to 30, wherein said composition is injected through a needle, catheter or trocar.

32. The composition of any one of claims 1 to 10 and 25 to 30, wherein said composition is injected during the course of an endoscopic procedure.

33. The composition of any one of claims 1 to 10 and 25 to 30, wherein said composition is injected percutaneously.

34. The composition of any one of claims 1 to 10 and 25 to 30, wherein said composition is pre-heated at a temperature between 20 and 45° C. before being injected.

35. A method for treating urinary incontinence comprising the step of injecting a composition as defined in any one of claims 1 to 10 and 25 to 30 into an area of the urethral sphincter, said composition having a bulking action into said sphincter, thus treating urinary incontinence.

36. A method for breast augmentation comprising the step of injecting a composition as defined in any one of claims 1 to 10 and 25 to 30 into a breast, said composition increasing the tissue volume of the breast.

37. A method of cosmetic treatment of wrinkles comprising the step of injecting a composition as defined in any one of claims 1 to 10 and 25 to 30 into a soft tissue in or around the face of a patient, said composition having a cosmetically acceptable consistency for providing a mechanical support or a volume or thickness increase to surrounding soft tissues of the patient.

38. A method of load bearing tissue augmentation comprising the step of injecting a composition as defined in any one of claims 1 to 10 and 25 to 30 between said load bearing tissue and a load-exerting medium.

39. A method for treating acne scars or viral pock marks comprising the step of injecting a composition as defined in any one of claims 1 to 10 and 25 to 30 into a soft tissue underlying said scar or pock mark of a patient for increasing the volume of the soft tissue masking the scars or the pock marks.

40. A method for changing the contour of a nose of a patient comprising the step of injecting a composition as defined in any one of claims 1 to 10 and 25 to 30 into the soft tissue on the contour of the nose for increasing the volume of the soft tissue, thus changing the contour of the nose.

41. A method for augmenting the volume or thickness of soft-tissues comprising the step of injecting a composition as defined in any one of claims 1 to 10 and 25 to 30 into a soft tissue.

42. A method for operating plastic corrections comprising the step of injecting a composition as defined in any one of claims 1 to 10 and 25 to 30 into a soft tissue or a tissue cavity to create the plastic corrections.

43. A method for operating reconstructive or restorative surgeries comprising the step of injecting a composition as defined in any one of claims 1 to 10 and 25 to 30 into a soft tissue, a body cavity or conduit, an organ wall or a part thereof to create a reconstructive or restorative action.
